(19) United States
(12) Patent Application Publication
(10) Pub. No.: US 2007/0105766 A1
(43) Pub. Date: May 10, 2007

(54) Modulators of the abc transporter family and methods for their use

(76) Inventors: Bruce Staton, Hanover, NH (US); George A. O'Toole JR., Hanover, NH (US); Agnieszka Swiatecka-Urban, Hanover, NH (US); Daniel P. MacEachran, Orford, NH (US); Sophie Moreau Marquis, Nashua, NH (US)

Correspondence Address: LICATA & TYRRELL P.C., 66 E. MAIN STREET, MARLTON, NJ, 08053, US

(21) Appl. No.: 10/575,577

(22) PCT Filed: Oct. 14, 2004
(86) PCT No.: PCT/US04/33874

§ 371 Date: May 24, 2006

Related U.S. Application Data
(60) Provisional application No. 60/511,609, filed on Oct. 15, 2003.

(51) Int. Cl. A61K 38/16

(52) U.S. Cl. 514/12

(57) ABSTRACT

An isolated factor derived from the bacterium Pseudomonas aeruginosa that reduces expression of ABC transmembrane proteins and active fragments and mimetics thereof are provided. Also provided is a method for inhibiting expression of ABC transmembrane proteins in cells by administering to the cells the isolated factor or active fragment or a mimetic thereof. Such methods are useful in the enhancing delivery of small molecule therapeutic agents to the CNS and in treating cancers, particularly multidrug resistant cancers, and secretory diarrhea. In addition, compositions, methods for identifying compositions and methods for use of compositions which inhibit suppression of ABC transmembrane protein expression by this factor are provided. Such compositions and methods are useful in treatment of cystic fibrosis.

This patent application claims the benefit of priority from U.S. Provisional Patent Application Ser. No. 60/511,609, filed Oct. 15, 2003, the teachings of which are herein incorporated by reference in their entirety.

This invention was supported in part by funds from the U.S. government (NIH Grant No. P20-RR018787-01, NIH grant No. RO1 AI51360-01, NIH Grant No. RO1-DK45881, and NIH Grant No. RO1-DK34533). The U.S. government may therefore have certain rights in the invention.

Introduction

1. Field of the Invention

A factor, believed to be a protein, which is secreted by Pseudomonas aeruginosa has now been identified as reducing plasma membrane expression of ATP-binding cassette (ABC) transmembrane proteins such as P-glycoprotein (Pgp or multidrug resistance protein (MDR)), multidrug resistance associated protein 2 (MRP2), Cystic Fibrosis Transmembrane Conductance Regulator (CFTR), and other members of the ABC transporter family, whose functions include control of the transport of small molecules across cell membranes. Inhibition of the expression of these ABC transmembrane proteins in the plasma membrane by this newly identified factor or active fragments or mimetics thereof is expected to be useful in promoting delivery of therapeutics to the central nervous system, treating cancers that have developed resistance to conventional therapies due to over expression of multidrug resistance transporters, and in inhibiting secretory diarrhea. Suppression of the inhibitory effects of this newly identified factor on expression of ABC transmembrane proteins, particularly CFTR expression, is expected to be useful in treatment of patients with cystic fibrosis.

2. Background of the Invention

A family of proteins found on the surface of cells is known as the ATP-binding cassette (ABC) family of transmembrane proteins. Expression of these proteins affects the therapeutic accumulation of drugs in the central nervous system. This family of proteins includes, but is not limited to, the transmembrane ATP-dependent drug translocation protein P-glycoprotein (Pgp; Nooter, K. and Sonneveld, P. Leuk. Res. 1993 18:233-243; Biedler, J. L. Cancer Res. 1994 54:666-678; Kerbel et al. Cold Spring Harbor Symp. Quant. Biol. 1994 59:661-672; Broxterman et al. Curr. Opin. Oncol. 1995 7:532-540; and List, A. F. Leukemia 1996 10:937-942), also referred to as the Multi-drug Resistance Protein (MDR), whose over expression is associated with multi-drug resistance (Demolombe, S. and Escande, D. TIPS 1996 17:273-275); and multidrug resistance associated protein 2 or MRP2, and the chloride channel Cystic Fibrosis Transmembrane Conductance Regulator (CFTR).

Pgp is expressed in a variety of normal tissues including liver, kidney and colon and tumors arising from these tissues usually over express Pgp as part of their multi-drug resistance (MDR) phenotype (Cole et al. Science 1992 258:1650-1654; Roninson, I. B. Biochem. Pharmacol. 1992 43:95-102; Arceci, R. J. Blood 1993 81:2215-2222; and Merkel et al. J. Clin. Oncol. 1989 7:1129-1136). Pgp can also be over expressed in tumors from tissues that do not normally express this protein, such as breast and ovarian tissues (Arceci, R. J. Blood 1993 81:2215-2222; and Ihnat et al. Clin. Cancer Res. 1997 3:1339-1346). The mechanism of Pgp upregulation in tumors in vivo is still unclear, but can occur de novo as in acute myologenous leukemia (AML) (Gregorcyk et al. Ann. Surg. Oncol. 1996 3:8-14; Koh et al. Yonsei Medical Journal 1992 33:137-142; Dalton, W. S. and Sikic, B. I. J. NIH Res. 1994 6:54-58; Cole et al. Science 1992 258:1650-1654; Demolombe, S. and Escande, D. TIPS 1996 17:273-275; Schneider et al. British J. Cancer 1989 60:815-818; Fojo et al. Proc. Natl. Acad. Sci. USA 1987 84:265-269; Roninson, I. B. Biochem. Pharmacol. 1992 43:95-102; Arceci, R. J. Blood 1993 81:2215-2222; and Merkel et al. J. Clin. Oncol. 1989 7:1129-1136) or can be acquired over the course of cancer treatment as in breast and ovarian cancer (Merkel et al. J. Clin. Oncol. 1989 7:1129-1136; Ihnat et al. Clin. Cancer Res. 1997 3:1339-1346; Hamilton, J. W. and Wetterhahn, K. E. Mol. Carcinogens 1989 2:274-286; and McCaffrey et al. Mol. Carcinogens 1994 10:189-198).

MDR1 gene transcription and MDR1 mRNA expression can be induced by certain DNA damaging agents, including chemotherapeutic drugs such doxorubicin, alkylating agents such as methyl methanesulfonate, and genotoxic chemical carcinogens that induce bulky DNA adducts such as aflatoxin B1 and 2-acetylaminofluorene.

MRP2 is also a member of the ABC family of transmembrane proteins involved in the transport of small molecules across cell membranes.

CFTR, another member of the ABC family of transport proteins is a cAMP-regulated chloride channel that mediates transepithelial chloride transport in the airways, intestine, pancreas, testis and other tissues. Cystic fibrosis, a lethal genetic disease, is caused by mutations in the CFTR gene, the most common of which is ΔF508. Intestinal CFTR contributes to the massive fluid and electrolyte losses in secretory diarrhea (Al-Awqati, Q. J. Clin. Invest. 2002 110(11):1599-601; Kunzelman et al. Physiol. Rev. 2002 82(1):245-89).

The ability to modulate the expression of these proteins has broad applications in a variety of clinical situations including, but not limited to, prevention of multidrug resistance in cancer, delivery of therapeutics to the central nervous system, inhibition of secretory diarrhea, and treatment of cystic fibrosis.

### SUMMARY OF THE INVENTION

An object of the present invention is to provide an isolated factor believed to be a protein, or an active fragment thereof, derived from the bacterium Pseudomonas aeruginosa that reduces expression of ABC transmembrane proteins in the plasma membrane.

Another object of the present invention is to provide a composition comprising a mimetic of the isolated factor, or an active fragment thereof, derived from the bacterium Pseudomonas aeruginosa that reduces expression of ABC transmembrane proteins in the plasma membrane for use in combination therapies for CNS disorders, infections and diseases and cancers exhibiting multidrug resistance.

Another object of the present invention is to provide a method for modulating plasma membrane expression of an ABC transmembrane protein in a cell comprising administering to the cell the isolated factor, or active fragment thereof, derived from Pseudomonas aeruginosa or a mimetic thereof that modulates plasma membrane expression of ABC transmembrane proteins.

Another object of the present invention is to provide a method for delivering a small molecule therapeutic agent to the central nervous system of a subject which comprises administering to the subject the isolated factor or active fragment thereof derived from Pseudomonas aeruginosa or a mimetic thereof that reduces expression of ABC transmembrane proteins in the plasma membrane so that expression of ABC transmembrane proteins which prevent small molecules from accumulating in the central nervous system is inhibited in the subject and administering to the subject the small molecule therapeutic agent.

Another object of the present invention is to provide a method for treating cancer in a subject which comprises administering to the subject the isolated factor or active fragment thereof derived from Pseudomonas aeruginosa or a mimetic thereof that reduces plasma membrane expression of ABC transmembrane proteins so that expression of ABC transmembrane proteins which confer drug resistance in cancer cells is inhibited in the subject and administering to the subject an anti-cancer agent. This method of treating cancer is particularly useful in cancers that have become resistant to therapy due to overexpression of ABC transmembrane proteins and/or to cancers of the central nervous system.

Another object of the present invention is to provide a method for treating secretory diarrhea in a subject which comprises administering to the subject the isolated factor or active fragment thereof derived from Pseudomonas aeruginosa or a mimetic thereof that reduces plasma membrane expression of ABC transmembrane proteins, and in particular intestinal CFTR expression, so that massive fluid and electrolyte losses in secretory diarrhea are inhibited.

Another object of the present invention is to provide agents and methods for identifying agents which inhibit or suppress this Pseudomonas aeruginosa and active fragments thereof and their inhibitory effects on expression of ABC transmembrane proteins, particularly CFTR expression.

Yet another object of the present invention is to provide compositions and methods for treating or alleviating symptoms of a subject suffering from cystic fibrosis via administration of a composition comprising an agent which inhibits or suppresses this Pseudomonas aeruginosa factor and its inhibitory effects on expression of ABC transmembrane proteins, particularly CFTR expression.

### DETAILED DESCRIPTION OF THE INVENTION

A factor, believed to be a protein, which is secreted by clinical and laboratory isolates of the bacterium Pseudomonas aeruginosa has now been identified and partially purified that reduces or otherwise modulates the plasma membrane expression of CFTR as well as P-glycoprotein (i.e. Pgp or multidrug resistance protein (MDR)), MRP2 and other members of the ABC transmembrane protein family, whose functions include the transport of small molecules across cell membranes. More specifically, members of the ABC transporter family function by preventing therapeutics including but not limited to antibiotics from accumulating in the central nervous system, and by conferring the observed “drug resistance” of numerous cancers.

The factor of the present invention that reduces or otherwise modulates the plasma membrane expression of the ABC transmembrane protein family is secreted by P. aeruginosa during the stationary phase. This factor has been found to be heat-labile. The factor is retained by a membrane with a reported molecular weight cut-off of greater than 30 kDa. The factor of the present invention remains in solution in the presence of 70% ammonium sulfate and it interacts with the anion exchange resin at pH 6.5. Thus, the factor is believed to be a protein. The factor has been partially purified with ion exchange chromatography, ammonium sulfate precipitation, and gel filtration.

Studies were conducted demonstrating that P. aeruginosa affects the apical membrane expression of ABC transporters including CFTR, Pgp, and MRP2 in polarized epithelial cells. In these experiments two human cell lines endogenously expressing CFTR, Pgp, and MRP2, and polarized canine and rat cells stably expressing wild-type (wt)-CFTR and/or Pgp, were contacted with a pelleted fraction of either P. aeruginosa strain PA14 or eight different clinical isolates. Apical membrane expression of CFTR, Pgp, and MRP2 was measured by cell-surface biotinylation as set forth in Example 3.

P. aeruginosa strain PA14 (PA14, 5×10⁷ CFU/ml) reduced by >80% the apical membrane expression of endogenous CFTR in human cells and canine cells expressing wt-CFTR compared to heat-killed PA14 or vehicle alone as well. PA14 also decreased by >80% the apical membrane expression of wt-CFTR in rat cells. The effect of PA14 was not general for all plasma membrane proteins because PA14 had no effect on the apical membrane expression of glycoprotein gp114 or on the basolateral membrane expression of Na-K-ATPase or the transferrin receptor. All eight clinical isolates of P. aeruginosa tested also decreased the apical plasma membrane expression of wt-CFTR in canine cells.

To determine whether PA14 reduces apical membrane expression of CFTR via direct or indirect interactions with epithelial cells, apical membranes in the human, canine and rat cells were incubated with the cell-free PA14 filtrate containing factors secreted by PA14. The PA14 filtrate decreased the apical membrane expression of CFTR by >80% in all cell lines tested in 15 minutes compared to heat-inactivated PA14 filtrate or vehicle alone.

PA14 (5×10⁷ CFU/ml) and the cell-free PA14 filtrate containing the factors secreted by PA14 decreased by >70% the apical membrane expression of endogenous Pgp in both human cell lines. Similar results were observed in canine cells stably expressing Pgp. PA14 filtrate decreased by >70% the apical membrane expression of endogenous MRP2 in human cells compared to vehicle control as well.

Taken together, these observations support the conclusion that a heat-labile factor secreted by P. aeruginosa reduces the apical membrane expression of ABC transporters including CFTR, Pgp, and MRP-2 in polarized epithelial cells.

The ability of this secreted factor to inhibit expression of these transporters in the plasma membrane is indicative of its utility in facilitating delivery of small molecule therapeutic agents to the central nervous system, for enhancing efficacy of antibiotics as well as anti-cancer agents, particularly in multidrug resistant cancers and cancers of the central nervous system, and inhibiting massive fluid and electrolyte losses in secretory diarrhea.

While experiments to date have been performed using a partially purified factor believed to be a protein from clinical and laboratory isolates of the bacterium Pseudomonas aeruginosa, as will be understood by those of skill in the art upon reading this disclosure, the factor can also be prepared recombinantly or synthetically. Accordingly for purposes of the present invention, by the phrase “an isolated factor or protein derived from the bacterium Pseudomonas aeruginosa” it is meant to encompass the secreted factor, believed to be a protein, purified or partially purified from the bacterium as well as proteins having the same amino acid sequence as this factor but which are prepared recombinantly or synthetically using well known techniques.

Further, fragments of this factor may exhibit similar inhibitory activities. Such fragments are referred to herein as active fragment or active peptide fragments. By “active fragment” or “active peptide fragment” it is meant a peptide shorter in amino acid sequence than the full-length factor but which exhibits the same or similar activity as an inhibitor of transmembrane receptor protein expression. Active fragments can be isolated from the bacterium or prepared recombinantly or synthetically in similar fashion to the full-length factor.

The identification of this factor, believed to be a protein, and its function as an inhibitor of transmembrane protein expression also enables the development of mimetics of this factor or active fragments thereof with similar ability to reduce expression of ABC transmembrane proteins in cells. By “mimetic”, as used herein it is meant to encompass peptidomimetics as well as small organic molecules similar in structure and/or inhibitory function to the factor or active fragments of the factor isolated from Pseudomonas aeruginosa.

Factors and active fragments thereof derived from the bacterium Pseudomonas aeruginosa that reduce expression of ABC transmembrane proteins or mimetics thereof can be administered to cells to inhibit the expression of an ABC transmembrane protein in the cells.

Inhibiting expression of an ABC transmembrane protein is expected to be useful in facilitating delivery of small molecule therapeutic agents to the central nervous system of a subject. In this method, the isolated factor or active fragment or mimetic thereof is first administered to the subject so that plasma membrane expression of an ABC transmembrane protein which prevents small molecules from entering the central nervous system is inhibited. The small molecule therapeutic agent to be delivered to the central nervous system can then be administered systemically to the subject without inhibition of accumulation of the therapeutic agent in the central nervous system by ABC transporter proteins. Examples of small molecule therapeutic agents which can be delivered to the central nervous system by this method include, but are not limited to, antibiotics and anti-cancer agents.

Inhibiting plasma membrane expression of an ABC transmembrane protein is also expected to be useful in enhancing efficacy of cancer treatments, particularly in cancer of the central nervous system and multidrug resistant cancers. In this method, a subject is administered the isolated factor or active fragment or a mimetic thereof so that expression of an ABC transmembrane protein which prevents small molecules from accumulating in the CNS or which confers drug resistance in cancer cells is inhibited in the subject. The anti-cancer agent is then administered to the subject.

The factor of the present invention secreted by P. aeruginosa works quickly within approximately 5 to 10 minutes. Accordingly, it is believed that a therapeutic agent can be delivered simultaneously or within 5 to 10 minutes of administration of the factor, active fragment thereof or the mimetic thereof.

The demonstrated ability of this factor to decrease transmembrane CFTR expression is also indicative of its utility in treating secretory diarrhea in subject. In particular, it is expected that administration of the isolated factor or active fragment thereof derived from P. aeruginosa or a mimetic thereof which reduces expression of ABC transmembrane proteins, and in particular intestinal CFTR expression, will inhibit the massive fluid and electrolyte losses from CFTR in secretory diarrhea.

By “subject” as used herein, it is meant to be inclusive of any mammal, including but not limited to humans.

In this embodiment, the factor, active fragment thereof, or mimetic thereof is preferably administered intravenously in a formulation acceptable for intravenous administration and in an amount sufficient to inhibit expression of ABC transmembrane proteins. However, as will be understood by those of skill in the art, alternative modes of administration and formulations acceptable for these alternative modes of administrations are within the scope of this invention.

Further, this heat-sensitive factor secreted by P. aeruginosa has been found to reduce CFTR-mediated, transepithelial Cl— secretion across polarized human airway epithelial cells in cells expressing ΔF508-CFTR, the most common mutation in cystic fibrosis.

These experiments were conducted in CFBE41o- cells stably expressing either wt-CFTR or ΔF508-CFTR and in parental (non-transfected ΔF508/ΔF508) CFBE41o- cells. To increase apical membrane expression of ΔF508-CFTR, cells were grown at 27° C. for 36 hours in accordance with methods set forth in Example 4. To control for any possible effects of reduced temperature on the results, parental and wt-CFTR expressing cells were also grown at 27° C. for 36 hours. As determined by cell surface biotinylation, reduced temperature had no effect on the apical membrane expression of wt-CFTR but significantly increased plasma membrane expression of ΔF508-CFTR. Parental CFBE41o- cells, and CFBE41o- cells stably expressing either wt-CFTR or ΔF508-CFTR were incubated at 27° C. in a CO₂ incubator in the absence of antibiotics. Vehicle or PA14 bacteria (5×10⁸ CFU/ml) were added to the apical side of the monolayers. PA14 inhibited the glybenclamide-sensitive, forskolin and genistein stimulated Isc in CFBE41o- stably expressing wt- and ΔF508-CFTR after 4-6 hours of incubation. The effect was not observed after exposing the apical side of the monolayers to PA14 for <3 hours. The parental CFBE41o- cells had no detectable glybenclamide-sensitive Isc before or after stimulation with genistein or forskolin, and PA14 had no effect on the Isc either before or after addition of genistein or forskolin. Taken together these observations confirm that P. aeruginosa inhibits CFTR-mediated C1-secretion in polarized human airway epithelial cells expressing wt-CFTR, and extend these observations to reveal that PA14 also inhibits ΔF508-CFTR-mediated Cl— secretion in polarized, human airway epithelial cells.

Experiments were then performed to determine whether the inhibition of Isc was reversible. In these experiments CFBE41o- cells stably expressing either wt-CFTR or ΔF508-CFTR were first incubated with vehicle or PA14 bacteria as described supra. After 6 hours of incubation, the monolayers were washed and subsequently incubated at 37° C. in a CO₂ incubator with sterile media containing antibiotics. One hour after washing off the bacteria from the apical side of CFBE41o- monolayers the glybenclamide-sensitive, forskolin and genistein stimulated Isc recovered to control values. These observations indicate that P. aeruginosa reversibly inhibits CFTR-mediated Cl— secretion in intact, polarized human airway epithelial cells.

The effects of P. aeruginosa on CFTR mediated C1-secretion across polarized MDCK (kidney) cells stably expressing wt- or ΔF508-CFTR were examined as well. It was found that addition of PA14 (4-6 hour incubation at 37° C. with 5×10⁶ CFU/ml washed PA14 bacteria added to the apical solution) inhibited CPT-cAMP stimulated Isc in the wt- and ΔF508-CFTR expressing cells. Taken together these observations demonstrate that P. aeruginosa inhibited CFTR-mediated Cl— secretion in polarized human airway epithelial cells and polarized kidney epithelial cells stably expressing wt- or ΔF508-CFTR.

Experiments were then performed demonstrating that PA14 reversibly inhibits Isc across polarized human airway epithelial cells at least in part by decreasing the number of CFTR channels in the apical plasma membrane.

To determine whether P. aeruginosa decreased the expression of CFTR in the apical plasma membrane, polarized MDCK cells were incubated with vehicle or PA14 bacteria (5×10⁶ CFU/ml added to the apical medium in the absence of antibiotics) and the apical membrane expression of CFTR was measured by cell surface biotinylation. PA14 decreased the apical membrane expression of CFTR after 4-6 hours of incubation compared to vehicle or heat-killed PA14. The effect was not observed after exposing the apical side of the monolayers to PA14 for ≦3 hours. PA14 did not affect the expression of gp114 in the apical plasma membrane or the expression of either the Na, K-ATPase or the transferrin receptor expressed in the basolateral plasma membrane. Expression of ΔF508-CFTR in the apical membrane of MDCK cells was too low to examine by cell surface biotinylation, thus, the effect of PA14 on the apical membrane expression of ΔF508-CFTR could not be measured. PA14 also decreased the expression of CFTR in the apical plasma membrane of Calu-3 cells and inhibited the expression of wt-CFTR and ΔF508-CFTR in the apical plasma membrane of CFBE41o- cells.

To determine whether this decrease in the expression of CFTR in the apical membrane was reversible, CFBE41o- cells stably expressing either wt-CFTR or ΔF508-CFTR were first incubated with vehicle or PA14 bacteria as described supra. After 6 hours of incubation, the monolayers were washed and subsequently incubated at 37° C. in a CO₂ incubator with sterile media containing antibiotics. One hour after washing off the bacteria from the apical side of CFBE41o- monolayers, CFTR expression in the apical membrane recovered to control values. These observations indicate that P. aeruginosa reversibly inhibits the apical membrane expression of CFTR in intact polarized human airway epithelial cells.

Experiments were then performed demonstrating that inhibition of CFTR expression in the apical membrane is mediated by the heat-sensitive factor secreted by P. aeruginosa described herein. In these experiments bacteria-free PA14 filtrate was prepared as described in Example 2. Vehicle or PA14 filtrate was added to the apical side of polarized MDCK monolayers, and the apical membrane expression of CFTR was measured by cell surface biotinylation. The PA14 filtrate rapidly (in minutes) decreased expression of CFTR in Calu-3 cells and inhibited the expression of wt-CFTR and ΔF508-CFTR in the apical membrane in CFBE41o- cells. Heating the PA14 filtrate resulted in loss of this activity.

This heat-sensitive factor secreted by P. aeruginosa was then demonstrated to inhibit recycling of CFTR from endosomes to the apical membrane. In these experiments, the endocytic recycling of CFTR was measured at 1, 3, and 5 minutes as described in Example 7. It was found that P. aeruginosa reduces the apical membrane expression of CFTR by rapidly inhibiting the recycling of CFTR from an endosomal pool back to the apical plasma membrane.

Data from these experiments provide direct evidence that P. aeruginosa inhibits the endocytic trafficking of CFTR in intact polarized human airway epithelial cells.

Cystic fibrosis patients are born with histologically normal lungs that become colonized with inhaled bacteria soon after birth, due to failure of the innate immunity of the airway. The early bacterial airway infection is accompanied by an intense neutrophilic inflammatory response in the peribronchial and endobronchial spaces. Subsequently, after several months to years, progressive obstructive pulmonary disease associated with chronic P. aeruginosa infection develops in approximately 80% of cystic fibrosis patients and, eventually, leads to respiratory failure and death. The density of P. aeruginosa in the airway increases with age of the cystic fibrosis patients. Colony counts of P. aeruginosa obtained in cystic fibrosis patients during bronchoscopy after lavaging the bronchus with 10-60 ml of sterile normal saline range from 10² to >10⁵ CFU/ml of the bronchalveolar lavage fluid. In direct quantitative sputum cultures from cystic fibrosis patients the colony counts are higher and range from 10⁷ to 10⁹ CFU/ml or gram of sputum. In cystic fibrosis patients the decline in pulmonary function correlates with the density of P. aeruginosa in the lower airway and is worst with ≧10⁵ CFU/ml bronchalveolar lavage fluid. In addition, in severely-ill non-cystic fibrosis patients with pneumonia, colony counts ≧10⁴ CFU/ml bronchalveolar lavage fluid may be present despite antibiotic treatment. Because P. aeruginosa decreases CFTR-mediated Cl— transport in polarized human airway epithelial cells at concentrations (5×10⁶-5×10⁸ CFU/ml) comparable to those described above in human airway, experiments described herein indicate that similar inhibition of Cl— transport may occur in the airway during infection with P. aeruginosa. Thus, it is believed that restoration of CFTR-mediated Cl— transport and mucociliary clearance by a combined therapy including: (1) promoting ΔF508-CFTR exit from the endoplasmic reticulum, (2) activating ΔF508-CFTR in the apical plasma membrane, and (3) increasing the half-life of ΔF508-CFTR in the apical membrane may be compromised by the presence of chronic and irreversible infection with P. aeruginosa. Based upon experiments described herein, however, it is expected that Cl— transport can be restored and the innate immunity in the airway reinstated in these patients by including in this therapy an additional agent which inhibits or suppresses the isolated Pseudomonas aeruginosa factor of the present invention and its inhibitory effects on expression of ABC transmembrane proteins, particularly CFTR expression.

Thus, the present invention also provides for agents and methods for identifying agents which inhibit or suppress the isolated Pseudomonas aeruginosa factor described herein. Such agents can be identified routinely by those skilled in the art based upon methodologies described herein for measuring CFTR expression in the presence of Pseudomonas aeruginosa or the isolated Pseudomonas aeruginosa factor. Inhibition of CFTR expression in cells in the presence of Pseudomonas aeruginosa or the isolated Pseudomonas aeruginosa factor can be measured as described herein in the presence and absence of a test agent. An increase in CFTR expression in the presence of a test agent is indicative of the agent being an inhibitor of suppression of CFTR expression by Pseudomonas aeruginosa or the isolated Pseudomonas aeruginosa factor.

The present invention also provides methods for inhibiting suppression of CFTR expression resulting from infection by Pseudomonas aeruginosa. In these methods, cells infected by Pseudomonas aeruginosa are administered an agent which inhibits the isolated factor of Pseudomonas aeruginosa described herein and its suppression of CFTR expression.

Examples of such agents include, but are in no way limited to, compounds which bind to the isolated factor of Pseudomonas aeruginosa thereby preventing it from suppressing CFTR expression such as ligands and antibodies and agents which decrease levels of the isolated factor of Pseudomonas aeruginosa such as antisense agents or targeted ribozymes.

Agents which inhibit the isolated Pseudomonas aeruginosa factor identified herein are expected to be useful in pharmacologic suppression of the effect of P. aeruginosa on CFTR. Thus, such agents are expected to be useful in treatment and/or alleviation of symptoms of subjects suffering from cystic fibrosis. These agents can be administered alone, or more preferably in combination with known therapies which: (1) promote CFTR exit from the endoplasmic reticulum, (2) activate CFTR in the apical plasma membrane, and/or (3) increase the half-life of CFTR in the apical membrane. By “in combination” it is meant that the agent is administered either simultaneously, before or after the other therapy for cystic fibrosis. Agents which inhibit the isolated Pseudomonas aeruginosa factor identified herein can be administered by various routes including, but in no way limited to, intravenously, intramuscularly, intraperitoneally, via inhalation, intranasally, intrabucally, and orally. Formulations comprising these agents can be prepared using well known techniques selected routinely by the skilled artisan in accordance with the route of administration.

The following nonlimiting examples are provided to further illustrate the present invention.

### EXAMPLES

### Example 1

### Cell Lines and Cell Culture

Two human cell lines endogenously expressing CFTR, Pgp, and MRP2: (1) Calu-3, an airway epithelial cell line and (2) Caco-2, an intestinal epithelial cell line, were used as a model of polarized epithelial cells. Studies were also conducted in human tracheal epithelial cells (CFBE410⁻) stably expressing either wt-CFTR or ΔF508-CFTR, Madin Darby Canine Kidney (MDCK) cells stably expressing either wt-CFTR or ΔF508-CFTR and Fisher Rat Thyroid (FRT) cells stably expressing wt-CFTR, and in MDCK cells stably expressing Pgp.

Epithelial cell cultures were grown on Transwell permeable growth supports (24 mm diameter, 0.4 μm pore size; Corning Corporation, Corning, N.Y.: #3412). Apical membrane expression of CFTR, Pgp, and MRP2 was measured by cell-surface biotinylation as described by Lisanti, et al. (J. Cell Biol. 1989 109:2117-2127) and Swiatecka-Urban et al. J. Biol. Chem. 2002 277(42):40099-105).

Calu-3 cells obtained from the American Type Culture Collection (Manassas, Va.) were maintained in MEM containing 50 U/ml penicillin, 50 μg/ml streptomycin, 2 mM L-glutamine, 1 mM sodium pyruvate, and 10% FBS in a 5% CO₂/95% air incubator at 37° C. Calu-3 cells were seeded on Transwell permeable supports (1×10⁶ on 6.5 mm and 4×10⁶ on 24 mm diameter, 0.4 μm pore size; Corning Corporation; Corning, N.Y.) coated with Vitrogen plating medium (VPM) containing DMEM (JRH Biosciences; Lenexa, Kans.), human fibronectin (10 μg/ml; Collaborative Biomedical Products; Bedford, Mass.), 1% Vitrogen 100 (Collagen; Palo Alto, Calif.), and BSA (10 μg/ml; Sigma-Aldrich; St. Louis, Mo.). Cells were grown in air-liquid interface culture at 37° C. for 14 to 21 days. Under these conditions, Calu-3 cells become polarized.

CFBE41o- parental cells (ΔF508/ΔF508), and CFBE41o- cells stably expressing either wt-CFTR or ΔF508-CFTR were maintained in MEM supplemented with 50 U/ml penicillin, 50 μg/ml streptomycin, 2 mM L-glutamine, 10% FBS, and 1 μg/ml blasticidine (wt-CFTR) or 2 μg/ml puromycin (ΔF508-CFTR) in a 5% CO₂/95% air incubator at 37° C. CFBE41o- cells were seeded on 12 mm Snapwell or 24 mm Transwell permeable supports (0.4 μm pore size; Corning Corporation; Corning, N.Y.) at 1×10⁶ and grown in air-liquid interface culture at 37° C. for 6-9 days and at 27° C. for 36 hours to increase trafficking and expression of ΔF508-CFTR in the apical membrane. Under these conditions, CFBE41o- cells form polarized monolayers.

MDCK cells stably expressing GFP-wt-CFTR or GFP-ΔF508-CFTR fusion proteins were established and maintained in culture in a 5% CO₂/95% air incubator at 37° C. in MEM complete medium containing 50 U/ml penicillin, 50 μg/ml streptomycin, 2 mM L-glutamine, 10% FBS, and 150 μg/ml G418. Addition of GFP to the N-terminus of CFTR has no effect on CFTR localization, trafficking, function as a Cl channel, or on its degradation. MDCK cells were seeded on Transwell permeable supports (0.2×10⁶ on 6.5 mm and 24 mm diameter, 0.4 μm pore size; Corning Corporation; Corning, N.Y.) and grown in culture at 37° C. for 7-10 days. Under these conditions, MDCK cells become polarized. Sodium butyrate (5 mM; Sigma-Aldrich; St. Louis, Mo.) was added to the MDCK cell culture medium 16-18 hours before experiments to stimulate CFTR expression.

### Example 2

### P. aeruginosa Isolates for Studies on Apical Membrane Expression of ABC Transporters in Epithelial Cells

Lysogeny broth (5 ml) was inoculated with P. aeruginosa strain UCBPP-PA14 (PA14), a relatively recent laboratory isolate from a burn patient, from a glycerol stock and incubated at 37° C. with rotation until the bacterial count reached OD=1.5 corresponding to a bacterial count of 5×10⁹ CFU/ml (14-18 hours of culture). Bacteria were harvested by centrifuging cultures at 4800×g for 10 minutes at 4° C. Subsequently, after washing with PBS, pH 7.4 at 4° C. to eliminate factors secreted into the extracellular environment, the bacteria were resuspended in PBS to a stock concentration of 5×10⁹ CFU/ml. In addition, 8 clinical isolates of P. aeruginosa, 4 from cystic fibrosis and another 4 from non-cystic fibrosis patients, were cultured as described above. Heat-killed bacteria, used as a control, were prepared by incubating the PBS-resuspended cultures at 95° C. for 10 minutes.

Bacteria-free P. aeruginosa filtrates were prepared by centrifugation of bacterial cultures, grown as described above, at 4800×g for 10 minutes at 4° C. Supernatants were harvested and filter-sterilized at 4° C. using a 0.2 μm filter resulting in bacteria-free filtrates. Heat-inactivated filtrates, used as control, were prepared by incubating the filtrates at 60° C. for 45 minutes.

### Example 3

### Measurement of Apical Membrane Expression of ABC Transporters in the Presence of P. aeruginosa Factor

In these experiments, apical membrane proteins were biotinylated at 4° C. using a derivative of biotin (EZ-Link™ Sulfo-NHS-LC-Biotin: Pierce). Subsequently, apical membranes in epithelial cells were incubated with washed P. aeruginosa strain PA14 or with cell-free PA14 filtrate at 37° C. Heat-killed PA14 (10 minutes at 90° C.), heat-inactivated PA14 filtrates (10 minutes at 60° C.), and vehicle alone (PBS or LB media) were used as control. Subsequently, cells were lysed, biotinylated proteins were isolated by streptavidin-agarose beads, eluted into SDS-sample buffer, and separated by 7.5% SDS-PAGE. Biotinylated CFTR, Pgp, and MRP-2 were analyzed by Western blotting using antibodies against CFTR, Pgp, and MRP-2, respectively. Ussing chamber studies that measure CFTR-mediated chloride currents were performed to provide additional, functional support for the view that PA14 reduced the plasma membrane expression of CFTR as described by Vandorpe et al. (Am. J. Physiol. Cell Physiol. 1995 269(38):C683-689).

### Example 4

### Ussing Chamber Measurements

Monolayers grown on Transwell (6.5 mm diameter) or Snapwell (12 mm diameter) permeable supports, as described in Example 1, were mounted in an Ussing-type chamber (Jim's Instruments; Iowa City, Iowa or Physiologic Instruments; San Diego, Calif.) and bathed in solutions, pH 7.4 maintained at 37° C. and stirred by bubbling with 5% CO₂/95% air. Short circuit current (Isc) was measured by voltage-clamping the transepithelial voltage across the monolayers to 0 mV with a voltage clamp (model VCC MC6, Physiologic Instruments, San Diego, Calif.). Current output from the clamp was digitized by an analog-to-digital converter (iWorx; Dover, N.H.). Data collection and analysis were done with LabScribe v1.6 Software (iWorx; Dover, N.H.). Stimulated Isc was measured after addition of 100 μM CPT-cAMP (Calu-3 and MDCK cells) or 20 μM forskolin (wt-CFTR CFBE41o- cells) to the apical and basolateral bath solution or 50 μM genistein (ΔF508-CFTR CFBE41o- cells) to the apical bath solution. Net stimulated Isc (ΔIsc) was calculated by subtracting the baseline Isc measured before stimulation from the peak Isc measured after stimulation. Glybenclamide (200 μM) was added to the apical bath solution to inhibit CFTR-mediated Isc.

Intact Calu-3 monolayers were bathed in MEM (-FBS). In order to determine the effects of PA14 on CFTR-mediated Cl currents across the apical membrane of Calu-3 cells, basolateral membranes were permeabilized with nystatin (200 μg/ml) and an apical-to-basolateral Cl— concentration gradient was established. A low Cl—, high-Na⁺, high-gluconate, basolateral bath solution was used to prevent cell swelling due to the increased basolateral Cl— permeability under these conditions. The basolateral bath solution contained (in mM) 115 Na-gluconate, 5 NaCl, 25 NaHCO₃, 3.3 KH₂PO₄, 0.8 K₂HPO₄, 1.2 MgCl₂₁ 1.2 CaCl₂₁ 10 glucose. The apical bath solution contained (in mM) 120 NaCl, 25 NaHCO₃, 3.3 KH₂PO₄, 0.8 K₂HPO₄₁ 1.2 MgCl₂₁ 1.2 CaCl₂, 10 mannitol. Mannitol was substituted for glucose in the apical bath solution to eliminate the contribution of the Na-glucose cotransporter to Isc. Successful permeabilization of the basolateral membrane was based upon the recording of a current consistent with the apical-to basolateral flow of negative charge. CFBE41o- cells were bathed in solutions with apical-to-basolateral Cl— gradient in the presence of amiloride (100 μM) in the apical bath solution to inhibit Na⁺ absorption through the epithelial Na⁺ channel (ENaC). The apical bath solution contained (in mM) 115 Na-gluconate, 5 NaCl, 25 NaHCO₃, 3.3 KH₂PO₄, 0.8 K₂HPO₄₁ 1.2 MgCl₂, 4 Ca gluconate, 10 mannitol. The basolateral bath solution contained (in mM) 120 NaCl, 25 NaHCO₃, 3.3 KH₂PO₄, 0.8 K₂HPO₄₁ 1.2 MgCl₂, 1.2 CaCl₂, 10 glucose. MDCK monolayers were bathed in MEM solution in the presence of amiloride (100 μM) in the apical bath solution to inhibit Na⁺ absorption through ENaC.

### Example 5

### Endocytic Assay and Endocytic Recycling Assay

Endocytic assays and endocytic recycling assays were performed on polarized epithelial cells in accordance with procedures described by Swiateck-Urban et al. (J. Biol. Chem. 2002 277(42):40099-105). Sodium butyrate (5 mM) was used to stimulate CFTR expression in Calu-3 and MDCK cells. The temperature in the incubator was reduced (27° C.) to increase apical membrane expression of wt-CFTR and ΔF508-CFTR in CFBE41o- cells.

### Example 6

### Antibodies

Monoclonal anti-gp114 antibody were obtained from the Université de la Mediterranée, Marseille, France. Other antibodies used were monoclonal anti-human CFTR C-terminus, clone 24-1 (R&D Systems; Minneapolis, Minn.), monoclonal anti-CFTR, clone M3A7 (Upstate Biotechnology; Lake Placid, N.Y.), monoclonal anti-GFP JL-8 (BD Biosciences; San Jose, Calif.), monoclonal anti-transferrin receptor (Zymed; San Francisco, Calif.), monoclonal anti-Na, K-ATPase (Upstate Biotechnology; Lake Placid, N.Y.), and goat anti-mouse and goat anti-rabbit HRP secondary antibodies (BioRad Laboratories; Hercules, Calif.). All purchased antibodies were used at the concentrations recommended by the manufacturer.

### Example 7

### Data Analysis and Statistics

Each experiment was repeated a minimum of three to six times. Statistical analysis of the data was performed using GraphPad Prism version 4.0 for Macintosh (GraphPad Software; San Diego, Calif.). Means were compared by t-test. A P value <0.05 was considered significant. Data are expressed as mean±SEM.

1. An isolated factor or active fragment thereof derived from the bacterium Pseudomonas aeruginosa that modulates plasma membrane expression of ABC transmembrane proteins.

2. A composition comprising a mimetic of the isolated factor or active fragment thereof of claim 1.

3. A method for modulating plasma membrane expression of an ABC transmembrane protein in a cell comprising administering to the cell the isolated factor or active fragment of claim 1.

4. A method for delivering a small molecule therapeutic agent to the central nervous system of a subject comprising:
(a) administering to the subject the isolated factor or active fragment of claim 1 so that expression of an ABC transmembrane protein which prevents small molecules from entering into or accumulating in the central nervous system is inhibited in the subject; and
(b) administering to the subject the small molecule therapeutic agent.

5. A method for treating cancer in a subject comprising:
(a) administering to the subject the isolated factor or active fragment of claim 1 so that expression of an ABC transmembrane protein which confers drug resistance in cancer cells is inhibited in the subject; and
(b) administering to the subject an anti-cancer agent.

6. The method of claim 5 wherein the cancer is resistant to therapy due to overexpression of ABC transmembrane proteins.

7. The method of claim 5 wherein the cancer comprises a cancer of the central nervous system.

8. A method for treating secretory diarrhea in a subject comprising administering to the subject the isolated factor or active fragment of claim 1 so that plasma membrane expression of intestinal CFTR is reduced and massive fluid and electrolyte losses in secretory diarrhea is inhibited.

9. A composition comprising an agent which inhibits suppression of plasma membrane expression of ABC transmembrane proteins by the isolated factor or active fragment thereof of claim 1.

10. The composition of claim 9 wherein the agent inhibits suppression of expression of CFTR.

11. A method for inhibiting suppression of CFTR expression in cells infected by Pseudomonas aeruginosa, said method comprising administering to the cells the composition of claim 10.

12. A method for treating or alleviating symptoms of a subject suffering from cystic fibrosis comprising administering to the subject the composition of claim 10.

13. The method of claim 12 further comprising administering to the subject a therapy which promotes CFTR exit from an endoplasmic reticulum, activates CFTR in an apical plasma membrane, or increases half-life of CFTR in an apical membrane.

14. A method for identifying an agent for treatment or alleviation of symptoms of cystic fibrosis comprising assessing a test agent's ability to inhibit suppression of CFTR expression by the isolated Pseudomonas aeruginosa factor of claim 1, wherein the ability of the test agent to inhibit suppression of CFTR expression by the isolated Pseudomonas aeruginosa factor is indicative of the agent being useful for treatment or alleviation of symptoms of cystic fibrosis.

15. A method for modulating plasma membrane expression of an ABC transmembrane protein in a cell comprising administering to the cell the mimetic of claim 2.

16. A method for delivering a small molecule therapeutic agent to the central nervous system of a subject comprising:
(a) administering to the subject the mimetic of claim 2 so that expression of an ABC transmembrane protein which prevents small molecules from entering into or accumulating in the central nervous system is inhibited in the subject; and
(b) administering to the subject the small molecule therapeutic agent.

17. A method for treating cancer in a subject comprising:
(a) administering to the subject the mimetic of claim 2 so that expression of an ABC transmembrane protein which confers drug resistance in cancer cells is inhibited in the subject; and
(b) administering to the subject an anti-cancer agent.

18. The method of claim 17 wherein the cancer is resistant to therapy due to overexpression of ABC transmembrane proteins.

19. The method of claim 17 wherein the cancer comprises a cancer of the central nervous system.

20. A method for treating secretory diarrhea in a subject comprising administering to the subject the mimetic of claim 2 so that plasma membrane expression of intestinal CFTR is reduced and massive fluid and electrolyte losses in secretory diarrhea is inhibited.
